SEQUENCE LISTING (5 sequences)

SEQ ID NO: 1 - DNA, 1374 bp, Homo sapiens
  CDS (1)..(1374)
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgacctcgaccgtcccggaaacgcaggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 2 - protein, 457 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRPRPSRKRRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - protein, 457 aa, Homo sapiens; identical to SEQ ID NO: 2
  PEPTIDE (1)..(457)

SEQ ID NO: 4 - DNA, 1374 bp, homo sapiens
  CDS (1)..(1374)
atgtggcagctgacatccctgctgctgttcgtggctacgtggggcatctcgggcacccctgccccgctggacagcgtgttctcctccagtgagcgtgcccaccaggtcctgcggatccgcaagcgggcgaattcctttctcgaagagctccgacactcgtcactcgagcgggagtgtatcgaggagatctgcgacttcgaagaggctaaggaaatcttccagaacgtggatgacaccctggcgttctggagcaagcacgtcgacggcgaccagtgtctggtgctccctctagagcacccctgtgcctcactctgctgcggccacggtacatgcatcgatggcatcggctcgttctcctgcgattgcaggtcaggctgggaggggagattctgccagcgtgaggtgtcgttcctgaactgttccctggacaatggcgggtgtacccactactgcctcgaggaagtgggctggagacgctgttcctgcgcccccggctacaagctgggcgatgacctgctccagtgccacccagcagttaagtttccctgtggccgcccttggaagagaatggagaagaagcgctcgcacctcaagaggccgcgcccctcccgcaaacgccgcctcatcgacggcaagatgacccggcgcggcgactccccttggcaggtggtcctgctggactccaagaagaagctggcctgcggagccgtgctgatccacccctcctgggtcctgacagccgcccactgcatggacgagtcgaagaagctcctagttaggctgggtgagtacgacctccgccgttgggagaagtgggagctcgacctggatattaaggaggtcttcgtgcaccctaactacagtaagtcgaccacagacaacgatatagcgcttctccatttggcccagccagctaccctgtcccagaccatcgtgccaatctgcctccccgactcgggcctggctgagagggagctcaaccaggccgggcaggagaccctggtcaccgggtggggctaccactcctcccgggagaaggaagctaagaggaaccgcacctttgtgctgaatttcatcaagattcctgtcgtgcctcacaacgagtgttcggaagtgatgtcaaacatggtctccgagaatatgctctgcgccggcattctgggggaccgccaggatgcttgcgagggggattccgggggtcccatggtcgcctctttccacggcacctggttcttagtgggactggtgtcctggggcgaaggctgcgggctgctccacaactacggcgtgtacaccaaggtttcacgctacctggattggatccatggacacatccgcgacaaagaggctccccagaagtcctgggccccctag

SEQ ID NO: 5 - protein, 457 aa, homo sapiens; identical to SEQ ID NO: 2

(19) United States
(12) Patent Application Publication
(10) Pub. No.: US 2009/0068721 A1
(43) Pub. Date: Mar. 12, 2009

(54) Process for the Production of Recombinant Activated Human Protein C for the Treatment of Sepsis

(75) Inventor: Villoo Morawala Patell, Bangalore (IN)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: AVESTHAGEN LIMITED, Bangalore (IN)

(21) Appl. No.: 11/914,751

(22) PCT Filed: May 24, 2006
(86) PCT No.: PCT/IB2006/001359

§ 371 Date: Apr. 21, 2008

(30) Foreign Application Priority Data
May 24, 2005 (IN) 626/CHE/2005

(51) Int. Cl. C12N 9/48

(52) U.S. Cl. 435/212

(57) ABSTRACT

The present invention relates to a recombinant method of production of activated Protein C. The invention relates to a method of construction, transformation, expression, purification and production of recombinant activated human protein C. DNA constructs comprising the control elements associated with the gene of interest has been disclosed. The nucleic acid sequence of interest has been codon optimized to permit expression in the suitable mammalian host cells.

### FIELD OF INVENTION

The present invention relates to a recombinant method of production of activated Protein C. The invention relates to a method of construction, transformation, expression, purification and production of recombinant activated human protein C. DNA constructs comprising the control elements associated with the gene of interest has been disclosed. The nucleic acid sequence of interest has been codon optimized to permit expression in the suitable mammalian host cells.

### BACKGROUND OF THE INVENTION

Xigris (Drotrecogin alfa) is a recombinant form of human Activated Protein C. It is a serine protease with the same amino acid sequence as human plasma derived Activated Protein C. Activated Protein C is an important modulator of the systemic response to infection and has anti-thrombotic, profibrinolytic and anti-inflammatory properties. Drotrecogin alfa (activated) is a glycoprotein of approximately 55 kD molecular weight. The precursor form of Protein C contains a pre pro leader peptide (absent in the mature protein), a γ-carboxyglutamic acid (Gla) domain of 9 Gla residues, a short helical hydrophobic amino acid stack, two epidermal growth factor (EGF)-like domains, a linking peptide between the light and the heavy chain, an activation peptide, and a trypsin-like SP domain in which the catalytic triad is located at His-211, Asp-257 and Ser-360. The main function of EGF-domain is to provide protein-protein or protein-cell interactions. The residues present in the EGF motif were also shown to functionally interact with different activators and substrates. In addition, the connecting helix has residues that participate in the coordination of calcium ion bound to the EGF-I domain that is envisaged to play a neuroprotective role.

Post translational modifications removes the di-peptide Lys-156-Arg-157, so that the single chain form is converted into a two-chain molecule linked by a di-sulphide bond. 80% of the zymogen PC is in this form. Also carboxylation of glutamic acid residues in the amino terminal Gla domain, hydroxylation of an Asp residue in the EGF-I domain and glycosylation are the other post-translational events. RhAPC and human plasma derived APC have the same sites of glycosylation, though some variations in the glycosylation structures exist. Human APC has four asparagine linked N-glycosylation sites. It has a five fold higher sialic acid compared to other plasma proteins.

Human APC has four asparagine linked N-glycosylation sites. It has a five fold higher fucose and a two fold higher sialic acid compared to other plasma proteins. Activated Protein C exerts by inhibiting Factors Va and VIII a. Invitro data indicate that Activated Protein C has indirect profibrinolytic activity through its ability to inhibit plasminogen activator inhibitor-1 (PAI-1) and limiting generation of activated thrombin-activatable-fibrinolysis-inhibitor. Additionally, in vitro data indicate that Activated Protein C may exert an anti-inflammatory effect by inhibiting human tumor necrosis factor production by monocytes, by blocking leukocyte adhesion to selectins, and by limiting the thrombin-induced inflammatory responses within the microvascular endothelium.

Several methods have been described for the expression of recombinant proteins in higher eukaryotic systems. CHO-K1, HEK293 (and variants) cell expression systems have now established themselves as the predominant systems of choice for mammalian protein expression. The procedure outlined is suitable for the transfection of the denovo synthesized nucleic acid sequence encoding the recombinant human Drotrecogin alfa into suitable mammalian hosts for expression.

The procedure outlined below is suitable for the production of bioactive, recombinant soluble recombinant activated human protein C. The current protocols make use of an established human cell line possessing the complementary DNA for the inactive human protein C zymogen that secrete the protein into the fermentation medium. Human Protein C is enzymatically activated by cleavage with alpha-thrombin, trypsin, Russell's viper venom factor X activator or a mixture of thrombin and thrombomodulin to obtain activated protein C and subsequently purified. However, these activation procedures involve the risk of contamination and higher costs of production. This investigation aims at the production of the activated protein C directly from the recombinant cells by the incorporation of the cell-associated protease.

Such proteases could be located in the cytoplasm or cell organelle or in the cell membranes that can cleave proteins during or immediately upon secretion. Accordingly, the strategy has been employed for the production of recombinant activated protein C directly upon secretion from a eukaryotic host cell namely HEK293.

The recombinant enzyme will be indicated for use in the reduction of mortality in adult patients with severe sepsis (i.e., sepsis associated with acute organ failure) who have a high risk of death.

### DESCRIPTION OF FIGURES INCLUDED

FIG. 1. Pair-wise sequence alignment of the non-optimized and codon-optimized versions of the DNA nucleotide sequence encoding Drotrecogin alfa or Xigris.

FIG. 2. Gel purified restriction-digested fragments of DROT cDNA, & pcDNA3.1D/V5-His

FIG. 3. Restriction digestion analysis of putative clones of AVCIPpcDNA3.1 D/V5-His/Xigris.

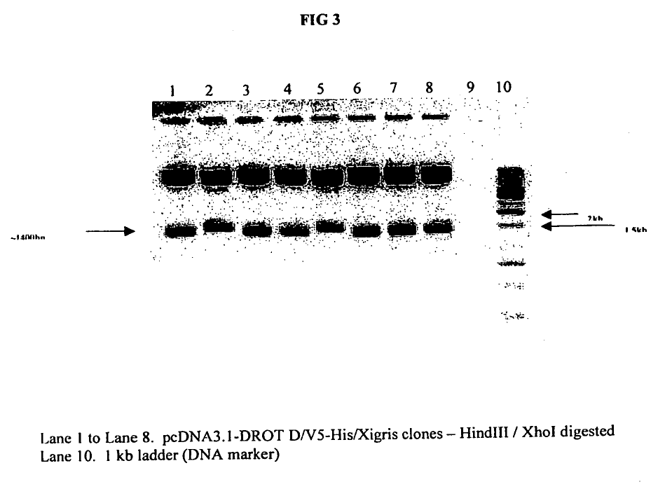

FIG. 4. Restriction digestion analysis of AVCIPpcDNA3.1D/V5-His/Xigris clones using enzymes that cleave pcDNA3.1-DROT cDNA internally

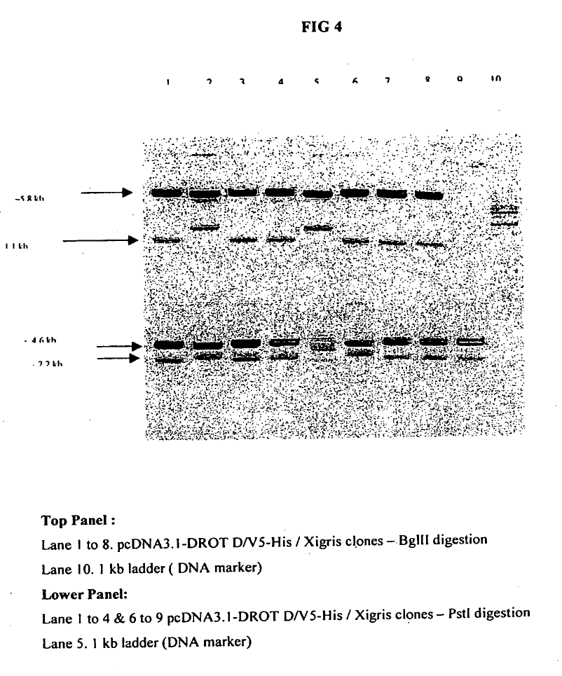

FIG. 5. Sequence alignment of the de novo synthesized pcDNA3.1-DROT (syntheticxigris) with the established sequence of the Xigris gene.

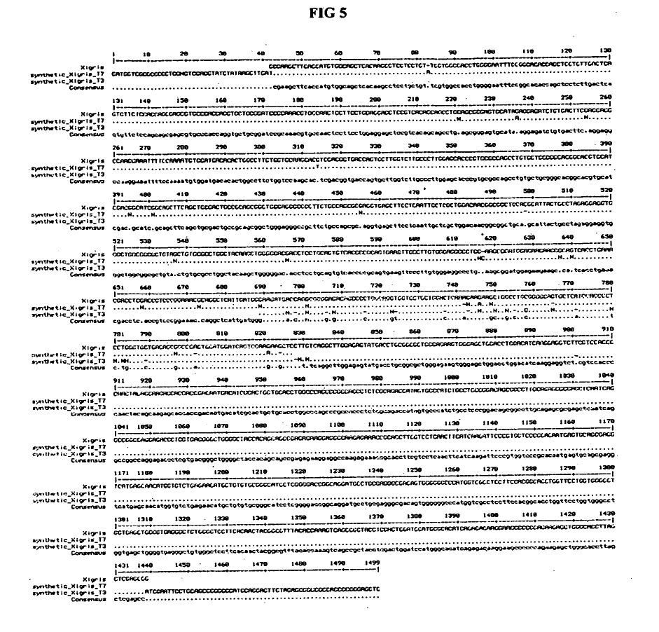

FIG. 6. Sequence alignment of the de novo synthesized pcDNA3.1-DROT-Opt (synthetic_Xigris-Opt) with the established sequence of the Xigris-Opt gene

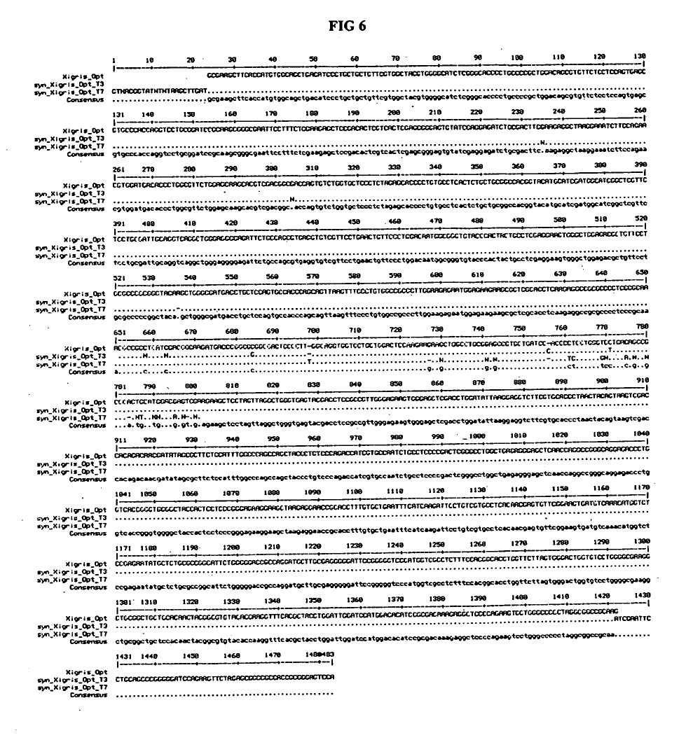

FIG. 7. Sequence alignment of pcDNA3.1DROT-/V5-His/Xigris cDNA clone # 4 with the established sequence of the Xigris gene

FIG. 8. Construct Map: pcDNA3.1-DROT-D/V5-His/Xigris

### SEQUENCE LISTINGS

SEQ ID NO 1: Nucleotide sequence of Activated Protein C
SEQ ID NO 2: Amino acid sequence of APC
SEQ ID NO 3: Codon optimized sequence of Activated Protein C

### SUMMARY OF THE INVENTION

DNA constructions comprising the control elements associated with the gene of interest which permit expression of the gene of interest has been disclosed. Still another aspect of the invention is the codon optimization of the denovo-synthesized nucleic acid to permit expression of the same in mammalian cells. The codon-optimized sequence is transformed into suitable mammalian cell lines for expression.

### DETAILED DESCRIPTION OF THE INVENTION

### Example I

The design of the mammalian expression vector for the expression of recombinant human protein C (activated) has been modified to accommodate four N-linked glycosylation sites and are be based on one of the commercially available vectors (EX: pcDNA or pIRES from Invitrogen or BD Biosciences respectively), modified to include the following features:
(a) A multiple cloning site for insertion of the human protein C cDNA including its natural signal peptide.
(b) The design of the expression vector also accommodates an independent (bi-cistronic) IRES-mediated co-expression of the green fluorescent protein which would allow rapid screening of highly expressing transfectants using fluorescence assisted cell sorting.

### Synthesis of the Fusion Construct:

### De Novo Approach:

A de novo approach in terms of synthesis of the coding regions of the rhAPC cDNA-construct has been pursued to enable better codon optimization with respect to the particular mammalian cell to be used. The design of the synthetic cDNA construct also include features such as:
   A Kozak consensus sequence (GCCACC) followed by, an initiation codon (ATG) to ensure efficient translation
   Suitable restriction sites at the 5′ and 3′ end of the cDNA to clone into the desired expression vector.

The nucleotide sequences the human activated protein C has been represented in SEQ ID: 1. The co dons in the coding DNA sequence of rhAPC that have been altered as part of the codon-optimization process to ensure optimal recombinant protein expression in mammalian cell lines such as CHO K1 and HEK 293. The codon optimized sequence of the nucleic acid has been depicted in SEQ ID NO: 2

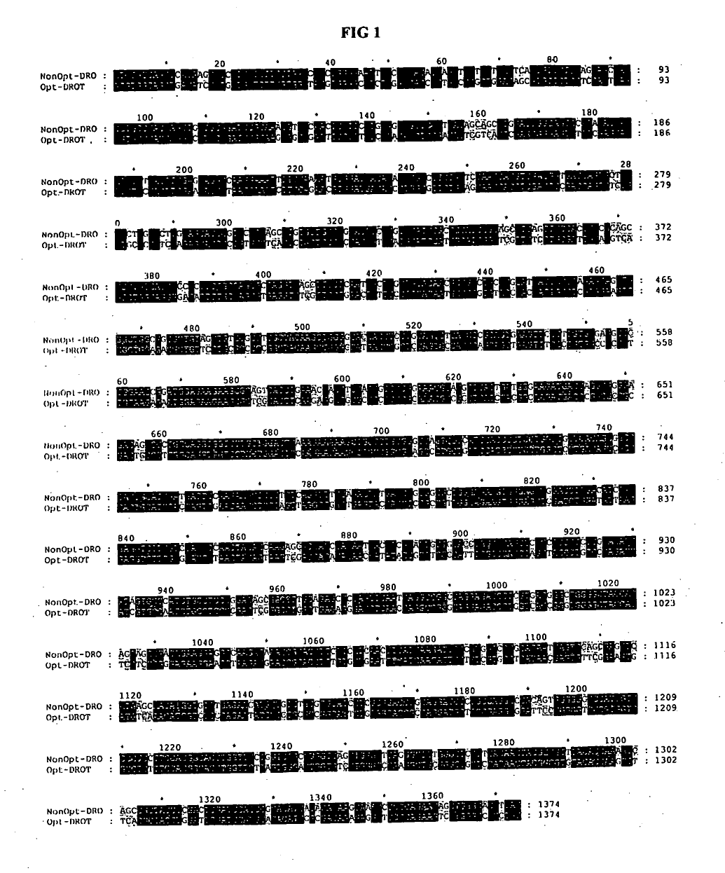

The optimized sequence of the nucleic acid sequence has been represented in SEQ ID: 2. Post codon optimization pair-wise sequence alignment of the non-optimized and codon-optimized versions of the DNA nucleotide sequence encoding Drotrecogin alfa or Xigris has been depicted in FIG. 1.

### Example 2

### Sub-Cloning of Drotrecogin ALFA (DROT) cDNA into the PCDNA3.1D/V5-His Mammalian Cell-Specific Expression Vector

Subsequent to the verification of the authenticity of the de novo synthesized cDNA molecules (DROT & DROT-Opt) by automated DNA sequencing as shown above, DROT was sub-cloned into the mammalian cell-specific expression vector pcDNA3.1D/V5-His to generate the transfection-ready constructs. The details of the procedures used are given below:

### A. Reagents and Enzymes

1. QIAGEN gel extraction kit & PCR purification kit
2. pcDNA 3.1D/V5-His vector DNA (Invitrogen)

| Enzyme | U/μl | 10x buffer |
|---|---|---|
| 1. HindIII | 10 | Buffer E |
| 2. XhoI | 10 | Buffer E |
| 3. T4 DNA ligase | 40 | Ligase Buffer |

### B. Restriction Digestion of the Vector and the Insert

Procedure

The following DNA samples and restriction enzymes were used:

| DNA samples | Restriction Enzyme |
|---|---|
| Rxn # 1 Vector (for Xigris cloning) | HindIII/XhoI |
| Rxn # 2 pBSK/ Xigris (#13) | HindIII/Xho I |

Restriction enzyme digest reaction:

| Components | Final conc. | Rxn #1 | Rxn #2 |
|---|---|---|---|
| Water | — | 4 μl | 4 μl |
| 10x Buffer | 1x | 2 μl | 2 μl |
| DNA | — | 10 μl | 10 μl |
| HindIII | 0.5 U | 1 μl | 1 μl |
| XhoI | 0.5 U | 1 μl | 1 μl |
| 10x BSA | 1x | 2 μl | 2 μl |
| Final volume | 20 μl | 20 μl | 20 μl |

The reaction was mixed, spun down and incubated for 2 hrs at 37° C. The restriction digestion was analyzed by agarose gel electrophoresis. The expected digestion pattern was seen. A gene fragment fall out of ˜1400 bp (for Rxn # 2) and a vector backbone fragment of ˜5.5 kb for Vector (Rxn # 1) was seen. The ˜1400 bp inserts of DROT & ˜5.5 kb digested vector pcDNA3.1 D/V5-His fragment were purified by gel extraction using the QIAGEN gel extraction kit. Checked 1 μl of the purified insert and vector fragment on a 1% agarose gel.

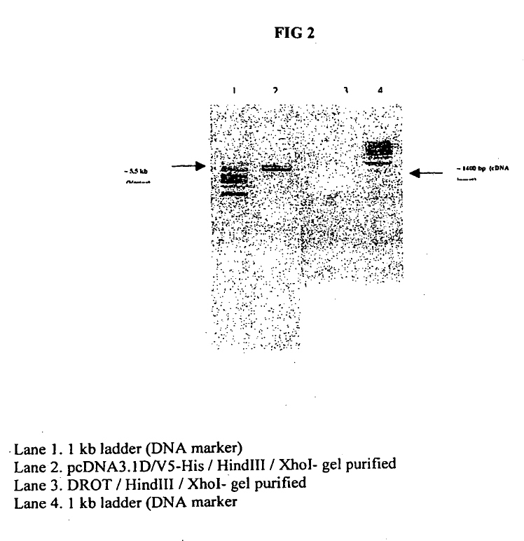

The gel purified restriction digested fragments of DROT cDNA and pcDNA3, 1D/V5-His has been represented in FIG. 2.

### Example 3

### C. Ligation of pcDNA3.1D/V5-His Backbone with DROT cDNA

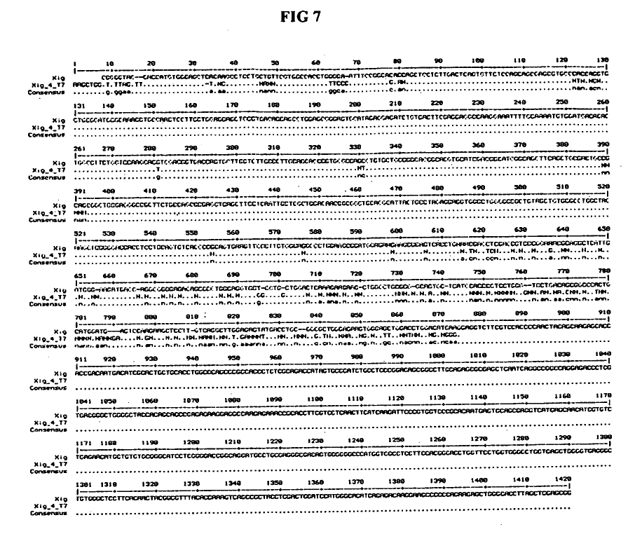

The DNA concentration of the digested & purified vector and insert fragments was estimated (ref. FIG. 7 above) and ligation was set up in the following manner:

| | | Rxn #1 | Rxn # 2 |
|---|---|---|---|
| Components | Final conc. | (Vector) | (Vector + Insert) |
| Water | — | 15 μl | 7 μl |
| 10xRxn buffer | 1x | 2 μl | 2 μl |
| Vector | ~50 ng | 2 μl | 2 μl |
| Insert | ~38 ng | — | 8 μl |
| T4 DNA ligase | 40 U | 1 μl | 1 μl |
| Final volume | 20 μl | 20 μl | 20 μl |

The reactions were gently mixed, spun down, and incubated at R.T, 2-3 hrs. DH10 competent cells were transformed with the ligation reactions.

The colonies obtained on L.B agar plates containing ampicillin were screened and confirmed by restriction digestion analysis of the isolated plasmid DNA.

### Example 4

### D. Restriction Digestion Analysis of Putative Clones of pcDNA3.1DROT-/V5-His/Xigris

Plasmid DNA was individually purified from the colonies obtained on L.B agar plates containing ampicillin and the presence of the desired cDNA insert was confirmed by restriction digestion analysis of the isolated plasmid DNA was undertaken. Restriction digestion analysis of the putative clones of AVC1PpcDNA3, iD/v5-His/Xigris has been represented in FIG.

In accordance with the results obtained after the restriction digestion of several putative clones containing the pcDNA3.1-DROT-D/V5-His/Xigris, some of the clones which showed the desired restriction pattern were selected for further restriction digestion analysis using restriction enzymes that cleave the AVCIP-Xigris cDNA internally to generate variable sized fragments as shown below in FIG. 9.

Restriction Digestion analysis of AVCiPpcDNA3, iD/V5-His/Xigris clones using enzymes that cleave pcDNA3.1-DROT cDNA internally.

Most of the pcDNA3.1-DROT D/V5-His/Xigris clones selected for the restriction mapping analysis yielded the expected fragment sizes based on the occurrence of known internal restriction sites and hence these clones were further verified by DNA sequencing analysis

### Example 5

### Verification of Authenticity of De Novo Synthesized cDNA Molecules

The verification of the authenticity of the de novo synthesized cDNA molecules as supplied by the commercial service provider was done by automated DNA sequencing

### E. Verification of Selected Clones of pcDNA3.1-DROT D/V5-His/Xigris by DNA Sequencing

The pcDNA3.1-DROT D/V5-His/Xigris clones selected as a result of the restriction mapping analysis were further verified by automated DNA sequencing.

| NOMENCLATURE | DESCRIPTION OF PRIMERS | SEQUENCES |
|---|---|---|
| T7 Sequencing | Invitrogen kit primer | 5′ TAATACGACTCACTATAGGG 3′ |
| primer | | |

pcDNA3.1-DROT D/V5-His/Xigris clone showed identity with the template sequence.

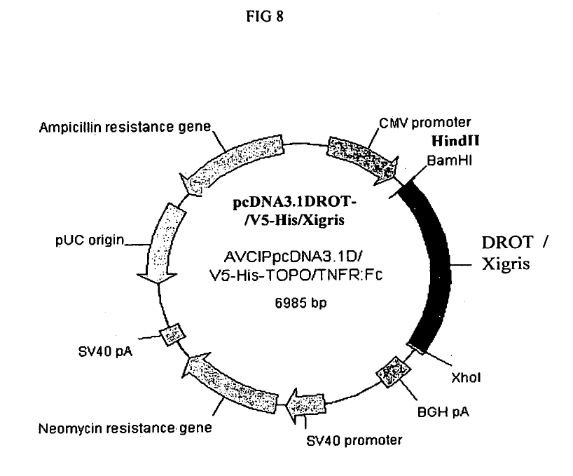

The map of the DROT is pictorially represented in the FIG. 8. recombinant expression construct made using the de novo synthesized pcDNA3.1—

### Example 6

### Maintenance and Propagation of the rhAPC Fusion Construct

The maintenance and propagation of the cDNA construct encoding rhAPC was done in a standard bacterial cell line such as Top 10 (Invitrogen).

### Example 7

### 5. Transient/Stable Recombinant Protein Expression in HEK293 Cells and Production of Supernatants

a) Transient/stable expression of the rhAPC construct was done using the human Embryonic Kidney cells (HEK293), transformed by sheared human adenovirus type 5 (AD 5) DNA which is a principal mammalian cell line that is FDA approved for industrial applications. Transient expression is useful to check the expression of a construct and to rapidly obtain small quantities of a recombinant protein.
b) Alternately, a protocol that allowed selection of large population of cells that exhibited high expression, rapidly, without having to obtain individual clones. Subsequently, HEK293 cells that displayed a stable and high expression of the desired rhAPC protein were developed using standard procedures.

Improved cultivation techniques using chemically defined culture media (Sigma Aldrich) as opposed to serum-containing media was used during the entire procedure in compliance with FDA requirements.

### Example 8

### Optimization of Purification Procedures

Subsequent to the establishment of reproducible bioactivity in accordance with the recommended functional/binding assays mentioned above, efforts will be made to optimize the purification procedures so as to maximize yield.

Accordingly, the purification process would comprise of the following downstream train:

a. Initial clarification and concentration using normal and tangential flow filtration procedures
b. Ultra filtration/Dialysis filtration (based on tangential flow filtration)
c. Chromo step-I: Affinity chromatography using monoclonal antibody to the activation site on the heavy chain of activated protein C or a calcium dependent antibody directed to the gamma carboxy glutamic acid domain of the light chain of human protein C.
d. Chromo step-II: Anion exchange chromatography using EMD fractogel
e. Chromo step-III: Flow through based anion exchangers such as cellufine sulfate for the removal of DNA and host cell proteins.
f. Virus removal and sterile filtration
g. Endotoxin removal
h. Formulation

1-6. (canceled)

7. A process for the preparation of an in vivo biologically active activated human protein C product comprising the steps of transforming a host cell with a synthesized DNA sequence encoding the protein encoded by the nucleic acid sequence of SEQ ID NO: 2 and isolating the product from the host cell or the medium of its growth.

8. The method according to claim 7, wherein the nucleic acid sequence encoding the activated human protein C has been represented in SEQ ID NO: 3.

9. The process according to claim 7, wherein the host cells are mammalian cells.

10. The process according to claim 7, wherein the host cells are from the strain HEK293.

11. A process for the preparation of an in vivo biologically active human recombinant activated protein C product comprising the steps of transforming a host cell with a vector construct of FIG. No. 8 and isolating the product from the host cell or the medium of its growth.

12. The process of claim 7, wherein the host cell is transformed with a vector as represented in FIG. NO: 8.
